(19) United States
(12) Patent Application Publication
(10) Pub. No.: US 2010/0137455 A1
(43) Pub. Date: Jun. 3, 2010

(54) N-VINYLCAPROLACTAM-BASED COPOLYMERS AND THE USE THEREOF AS SOLUBILIZERS

(75) Inventors: Nathalie Bouillo, Baden-Baden (DE); Marianna Pierobon, Ludwigshafen (DE); Gerhard Rossler, Neuhofen (DE); Rainer Dobrawa, Mannheim (DE); Kathrin Meyer- Boehm, Feucht (DE); Ronald Frans-Maria Lange, Ludwigshafen (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/063,492

(22) PCT Filed: Aug. 2, 2006
(86) PCT No.: PCT/EP06/64993

§ 371 Date: Feb. 11, 2008

(30) Foreign Application Priority Data
Aug. 11, 2005 (EP) 05107406.0

(51) Int. Cl. A61K 47/32; C08L 39/00; A23L 1/30

(52) U.S. Cl. 514/772.5; 524/548; 426/648

(57) ABSTRACT

Copolymers for use in solubilizing substances which are insoluble in water, substances which are only sparingly soluble in water, and combinations thereof; such copolymers comprising: (a) 60 to 99% by weight of N-vinylcaprolactam; and (b) 1 to 40% by weight of at least one other monomer selected from the group consisting of: C₈-C₃₀-alkyl esters of monoethylenically unsaturated C₃-C₈-carboxylic acids; N—C₈₋₃₀ alkyl-substituted amides and N,N—C₈₋₃₀ dialkyl-substituted amides of acrylic acid, methacrylic acid, or combinations thereof; vinyl esters of aliphatic unbranched C₈-C₃₀-carboxylic acids; C₈-C₃₀-alkyl vinyl ethers; and mixtures thereof.

The invention relates to the use of copolymers based on N-vinylcaprolactam as solubilizers of substances which are sparingly soluble in water. In addition, the invention relates to corresponding preparations for producing preparations for the treatment of humans, animals and plants, and also for industrial applications.

When producing homogeneous preparations of biologically active substances in particular, the solubilization of hydrophobic substances, i.e. substances which are sparingly soluble in water, has achieved very great practical importance.

Solubilization is understood as meaning making substances which are insoluble or sparingly soluble in a certain solvent, in particular water, soluble through interface-active compounds, the solubilizers. Such solubilizers are able to convert poorly water-soluble or water-insoluble substances into clear, at most opalescent aqueous solutions without the chemical structure of these substances undergoing a change in the process (cf. Römpp Chemie Lexikon, 9^{th} edition, Vol. 5, p. 4203, Thieme Verlag, Stuttgart, 1992).

The prepared solubilizates are notable for the fact that the poorly water-soluble or water-insoluble substance is present in colloidaily dissolved form in the molecular associates of the surface-active compounds which form in aqueous solution—the so-called micelles. The resulting solutions are stable single-phase systems which appear to be visually clear to opalescent and can be prepared without the input of energy.

Solubilizers can, for example, improve the appearance of cosmetic formulations and of food preparations by making the formulations transparent. Furthermore, in the case of pharmaceutical preparations, the bioavailability and thus the effect of medicaments can also be increased through the use of solubilizers.

The solubilizers used for pharmaceutical medicaments and cosmetic active ingredients are primarily surfactants such as ethoxylated (hydrogenated) castor oil, ethoxylated sorbitan fatty acid esters or ethoxylated hydroxystearic acid.

However, the hitherto used solubilizers described above have a number of application-related disadvantages.

The known solubilizers have only a small solubilizing effect for some sparingly soluble medicaments such as, for example, clotrimazole.

EP-A 876 819 describes the use of copolymers of at least 60% by weight of N-vinylpyrrolidone and amides or esters with long-chain alkyl groups.

EP-A 948 957 describes the use of copolymers of monoethylenically unsaturated carboxylic acids, such as, for example, acrylic acid, and hydrophobically modified comonomers, such as, for example, N-alkyl- or N,N-dialkylamides of unsaturated carboxylic acids with C₈-C₃₀-alkyl radicals.

A further desirable requirement of solubilizers is the ability to form so-called “solid solutions” with sparingly soluble substances. The term solid solutions refers to the state in which a substance is in molecularly disperse distribution in a solid matrix, for example a polymer matrix. Such solid solutions lead, for example when used in solid pharmaceutical administration forms of a sparingly soluble active ingredient, to an improved release of the active ingredient. An important requirement of such solid solutions is that they are stable even upon storage over a prolonged period, i.e. that the active ingredient should not crystallize out,

When forming solid solutions, besides the fundamental ability of the solubilizers to form solid solutions, the hygroscopicity of the solubilizers also plays an important role, Solubilizers which absorb too much water from the ambient air lead to deliquescence of the solid solution and to undesired crystallization of the active ingredients. Excessive hygroscopicity can also cause problems during processing to give administration forms.

The polymeric solubilizers known to date have the disadvantages that they either do not form stable solid solutions or are too hygroscopic. Furthermore, they still leave room for improvements with regard to solubilization in aqueous systems.

The object was therefore to provide novel and improved solubilizers for pharmaceutical, cosmetic, food and agrotechnical or other industrial applications which do not have the described disadvantages.

This object was achieved by using copolymers comprising
a) 60 to 99% by weight of N-vinylcaprolactam,
b) 1 to 40% by weight of at least one monomer chosen from the group of
b1) C₈-C₃₀-alkyl esters of monoethylenically unsaturated C₃-C₈-carboxylic acids,
b2) N-alkyl- or N,N-dialkyl-substituted amides of acrylic acid or of methacrylic acid with C₈ to C₃₀-alkyl radicals,
b3) the vinyl esters of aliphatic unbranched C₈-C₃₀-carboxylic acids,
b4) C₈-C₃₀-alkyl vinyl ethers,
   where the % by weight data of the individual components add up to 100% by weight.

in addition, the invention relates to preparations for substances which are sparingly soluble in water.

If appropriate, the copolymers can comprise 0 to 39% by weight of at least one further free-radically copolymerizable monomers c), where the % by weight data of the individual components a) to c) add up to 100% by weight.

The proportion of the monomer a) in the copolymer is preferably in the range from 70 to 95% by weight, particularly preferably in the range from 75 to 90% by weight.

Suitable monomers b) are:

N—C₈-C₃₀-alkyl- or N,N—C₈-C₃₀-dialkyl-substituted amides of monoethylenically unsaturated C₃-C₈-carboxylic acids, where the alkyl radicals are straight-chain or branched aliphatic or cycloaliphatic alkyl radicals having 8 to 30, preferably 8 to 18, carbon atoms. Suitable monoethylenically unsaturated carboxylic acids having 3 to 8 carbon atoms here are acrylic acid, methacrylic acid, dimethacrylic acid, ethacrylic acid, maleic acid, citraconic acid, methylenemalonic acid, allylacetic acid, vinylacetic acid, crotonic acid, fumaric acid, mesaconic acid and itaconic acid, preferably acrylic acid, methacrylic acid, maleic acid or mixtures of the specified carboxylic acids.

Preferred amidated comonomers are, for example, N-stearylacrylamide, N-stearyimethacrylamide, N-(1-methyl)undecylacrylamide, N-(1-methyl)undecylmethacrylamide, N-dodecylacrylamide, N-dodecylmethacrylamide, N-octylacrylamide, N-octylmethacrylamide, N,N-dioctylacrylamide, N,N-dioctylmethacrylamide, N-cetylacrylamide, N-cetylmethacrylamide, N-myristylacrylamide, N-myristylmethacrylamide, N-(2-ethyphexylacrylamide, N-(2-ethyl)hexylmethacrylamide.

In the case of maleic anhydride as comonomer, this can be reacted in a polymer-analogous manner with N-alkylamines by ring opening to give the corresponding amides.

Further comonomers b) which can be used are monoethylenically unsaturated C₃-C₈-carboxylic esters with a C₈-C₃₀-alcohol, preferably a C₈-C₁₈-alcohol.

Of particular importance in this connection are the acrylic and methacrylic esters with fatty alcohols with a chain length of from 8 to 18 carbon atoms, where the alkyl radicals may be branched or unbranched.

In particular, mention may be made here of: octyl acrylate, 2-ethylhexyl acrylate, nonyl acrylate, decyl acrylate, lauryl acrylate, myristyl acrylate, cetyl acrylate, stearyl acrylate, oleyl acrylate, behenyl acrylate, octyl methacrylate, 2-ethylhexyl methacrylate, nonyl methacrylate, decyl methacrylate, lauryl methacrylate, myristyl methacrylate, cetyl methacrylate, stearyl methacrylate, oleyl methacrylate, behenyl methacrylate, tert-butylcyclohexyl acrylate.

As further additional component b), vinyl esters of long-chain aliphatic, saturated or unsaturated, unbranched C₈-C₃₀-carboxylic acids, such as, for example, caprylic acid, capric acid, lauric acid, myristic acid, palmitic acid, stearic acid, arachidic acid, behenic acid, lignoceric acid, cerotinic acid, and melissic acid can be used.

In addition, as monomers b), C₈-C₃₀-alkyl vinyl ethers, preferably 0₈-C₁₈-alkyl vinyl ethers, can be copolymerized. Preferred alkyl radicals of the vinyl ethers which may be mentioned are branched or unbranched C₈-C₁₈-alkyl chains, such as, for example, n-octyl, 2-ethylhexyl, n-nonyl, n-decyl, n-undecyl, n-dodecyl, n-tridecyl, n-tetradecyl, n-pentadecyl, n-hexadecyl, n-heptadecyl and n-octadecyl.

Particularly preferred monomers b) are lauryl acrylate and vinyl laurate.

The proportion of the monomers b) is preferably 1 to 25% by weight, very particularly preferably 5 to 15% by weight.

Suitable additional free-radically copolymerizable monomers c) are:

monoethylenically unsaturated carboxylic acids having 3 to 8 carbon atoms or salts thereof, such as, for example, acrylic acid, methacrylic acid, dimethacrylic acid, ethacrylic acid, maleic acid, citraconic acid, methylenemalonic acid, allylacetic acid, crotonic acid, fumaric acid, mesaconic acid and itaconic acid.

From this group of monomers, preference is given to using acrylic acid, methacrylic acid or mixtures of the specified carboxylic acids.

The monoethylenically unsaturated carboxylic acids can be used in the copolymerization as free acid, as anhydrides, and also in partially or completely neutralized form.

For the neutralization of the abovementioned carboxylic acids, preference is given to using alkali metal or alkaline earth metal bases, ammonia or amines, preferably sodium hydroxide solution, potassium hydroxide solution, sodium carbonate, potassium carbonate, sodium hydrogen carbonate, magnesium oxide, calcium hydroxide, calcium oxide, gaseous or aqueous ammonia, triethylamine, ethanolamine, diethanolamine, triethanolamine, morpholine, diethylenetriamine or tetraethylenepentamine.

Further suitable comonomers c) are, for example, esters of monoethylenically unsaturated C₃-C₈-carboxylic acids with C₁-C₄-mono- or dialcohols or nitriles of said acids. Examples which may be mentioned are: methyl acrylate, ethyl acrylate, methyl methacrylate, ethyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, monomethyl maleate, dimethyl maleate, monoethyl maleate, diethyl maleate, acrylonitrile, methacrylonitrile. Further suitable comonomers c) are the N—C₁-C₄-alkyl- or N,N—C₁-C₄-dialkylamides of acrylic acid or of methacrylic acid, for example N-dimethylacrylamide or N-tert-butylacrylamide.

Also suitable are N,N—C₁-C₄-dialkylamino-C₁-C₄-alkyl acrylates, such as, for example, dimethylaminoethyl acrylate, diethylaminoethyl acrylate, dimethylaminoethyl methacrylate, diethylaminoethyl methacrylate, and the salts of the last-mentioned monomers with carboxylic acids or mineral acids, and also the quaternized products.

Further suitable monomers c) are, for example:

Acrylamidoglycolic acid, monoethylenically unsaturated sulfonic acids, such as vinylsulfonic acid, allylsulfonic acid, methallylsulfonic acid, styrenesulfonic acid, (3-sulfopropyl)acrylate, (3-sulfopropyl)methacrylate and acrylamidomethylpropanesulfonic acid;

monoethylenically unsaturated monomers comprising phosphonic acid groups, such as vinyiphosphonic acid, allyiphosphonic acid, acrylamidomethanepropanephosphonic acid.

In addition, as monomers c), the copolymers can also comprise vinyl acetate, N-vinylpyrrolidone, N-vinylimidazole, methylated N-vinylimidazole or N-vinylformamide.

It is of course also possible to use mixtures of the specified monomers.

Particularly preferred monomers c) are acrylic acid, methacrylic acid or itaconic acid and alkali metal salts thereof, very particularly preferably sodium acrylate.

The proportion of the monomer building blocks c) in the copolymer is preferably in the range from 0 to 15% by weight, particularly preferably it is 2 to 10% by weight.

The copolymers used according to the invention can have K values in accordance with Fikentscher, measured at 1% strength in 0.1 mol NaCl solution, of from 5 to 60, preferably 10 to 35, particularly preferably 12 to 30.

The copolymers are prepared by free-radically polymerizing the corresponding monomers.

The preparation is carried out by known processes, e.g. solution polymerization, precipitation polymerization or by inverse suspension polymerization using compounds which form free radicals under the polymerization conditions.

The polymerization temperatures are usually in the range from 30 to 200° C., preferably 40 to 110° C. Suitable initiators are, for example, azo and peroxy compounds, and the customary redox initiator systems, such as combinations of hydrogen peroxide and reducing compounds, e.g. sodium sulfite, sodium bisulfite, sodium formaldehyde sulfoxylate and hydrazine.

The reaction medium used is any customary solvent in which the monomers are soluble. Preference is given to using water or alcoholic solvents, such as, for example, methanol, ethanol, n-propanol or isopropanol or mixtures of such alcohols with water.

In order to ensure that the reaction leads to homogeneous products, it is advantageous to supply the monomers and the starter separately to the reaction solution. This can take place, for example, in the form of separate feeds for the individual reactants.

The polymerization can also be carried out in the presence of customary regulators if relatively low molecular weights are to be established.

The solids content of the organic solution obtained is usually 20 to 60% by weight, in particular 20 to 35% by weight.

A nonaqueous solvent used for the polymerization can then be removed by means of steam distillation and be replaced by water.

The aqueous solutions of the copolymers can, by various drying processes such as, for example, spray-drying, fluidized spray drying, drum drying or freeze-drying, be converted into powder form, from which an aqueous dispersion or solution can again be prepared by redispersion in water.

Applications:

The copolymers to be used according to the invention can in principle be used in all fields where substances which are insoluble or only sparingly soluble in water are either to be used in aqueous preparations or are to develop their effect in an aqueous medium. Accordingly, the copolymers are used as solubilizers of substances which are sparingly soluble in water, in particular biologically active substances.

According to the invention, the term “sparingly soluble in water” also comprises virtually insoluble substances and means that, for a solution of the substance in water at 20° C., at least 30 to 100 g of water per g of substance are required. In the case of virtually insoluble substances, at least 10 000 g of water per g of substance are required.

For the purposes of the present invention, biologically active substances which are sparingly soluble in water are understood as meaning pharmaceutical active ingredients for humans and animals, cosmetic or agrochemical active ingredients or food supplements or dietetic active ingredients.

In addition, suitable sparingly soluble substances to be solubilized are also dyes, such as inorganic or organic pigments.

By virtue of the present invention, amphiphilic compounds in particular for use as solubilizers for pharmaceutical and cosmetic preparations and also for food preparations are provided. They have the property of solubilizing sparingly soluble active ingredients in the field of pharmacy and cosmetics, sparingly soluble food supplements, for example vitamins and carotenoids, but also sparingly soluble active ingredients for use in crop protection compositions, and also veterinary medicine active ingredients.

Solubiilzers for Cosmetics:

According to the invention, the copolymers can be used as solubilizers in cosmetic formulations. For example, they are suitable as solubilizers for cosmetic oils. They have a good solubilizing ability for fats and oils, such as peanut oil, jojoba oil, coconut oil, almond oil, olive oil, palm oil, castor oil, soybean oil or wheatgerm oil or for essential oils, such as dwarf pine oil, lavender oil, rosemary oil, spruce needle oil, pine needle oil, eucalyptus oil, peppermint oil, sage oil, bergamot oil, terpentine oil, Melissa oil, juniper oil, lemon oil, anise oil, cardamom oil; peppermint oil, camphor oil etc. or for mixtures of these oils.

In addition, the polymers according to the invention can be used as solubilizers for UV absorbers which are insoluble or sparingly soluble in water, such as, for example, 2-hydroxy-4-methoxybenzophenone (Uvinul® M 40, BASF), 2,2′,4,4′-tetrahydroxybenzophenone (Uvinul® D 50), 2,2′-dihydroxy-4,4′-dimethoxybenzophenone (Uvinul® D49), 2,4-dihydroxybenzophenone (Uvinul® 400), 2′-ethylhexyl 2-cyano-3,3-diphenylacrylate (Uvinul®N 539), 2,4,6-trianilino-p-(carbo-2′-ethylhexyl-1′-oxy)-1,3,5-triazine (Uvinul® T 150), 3-(4-methoxybenzylidene)camphor (Eusolex® 6300, Merck), 2-ethylhexyl N,N-dimethyl-4-aminobenzoate (Eusolex® 6007), 3,3,5-trimethylcyclohexyl salicylate, 4-isopropyidibenzoylmethane (Eusolex® 8020), 2-ethylhexyl p-methoxycinnamate and 2-isoamyl p-methoxycinnamate, and mixtures thereof.

The present invention therefore also provides cosmetic preparations which comprise at least one of the copolymers according to the invention of the composition specified at the start as solubilizers. Preference is given to those preparations which, besides the solubilizer, comprise one or more sparingly soluble cosmetic active ingredients, for example the abovementioned oils or UV absorbers.

These formulations are solubilizates based on water or water/alcohol. The solubilizers according to the invention are used in the ratio from 0.2:1 to 20:1, preferably 1:1 to 15:1, particularly preferably 2:1 to 12:1 relative to the sparingly soluble cosmetic active ingredient.

The content of solubilizer according to the invention in the cosmetic preparation is, depending on the active ingredient, in the range from 1 to 50% by weight, preferably 3 to 40% by weight, particularly preferably 5 to 30% by weight.

In addition, further auxiliaries can be added to this formulation, for example nonionic, cationic or anionic surfactants, such as alkyl polyglycosides, fatty alcohol sulfates, fatty alcohol ether sulfates, alkanesulfonates, fatty alcohol ethoxylates, fatty alcohol phosphates, alkylbetaines, sorbitan esters, POE sorbitan esters, sugar fatty acid esters, fatty acid polyglycerol esters, fatty acid partial glycerides, fatty acid carboxylates, fatty alcohol sulfosuccinates, fatty acid sarcosinates, fatty acid isethionates, fatty acid taurinates, citric acid esters, silicone copolymers, fatty acid polyglycol esters, fatty acid amides, fatty acid alkanolamides, quaternary ammonium compounds, alkylphenol oxethylates, fatty amine oxethylates, cosolvents, such as ethylene glycol, propylene glycol, glycerol etc.

Further constituents which may be added are natural or synthetic compounds, e.g. lanolin derivatives, cholesterol derivatives, isopropyl myristate, isopropyl palmitate, electrolytes, dyes, preservatives, acids (e.g. lactic acid, citric acid).

These formulations are used, for example, in bath additive preparations such as bath oils, aftershaves, face tonics, hair tonics, eau de Cologne, eau de toilette and in sunscreen compositions. A further field of use is the oral care sector, for example in mouthwashes, toothpastes, adhesive creams for dentures and the like.

In addition, the copolymers are also suitable for industrial applications, for example for preparations of sparingly soluble colorants, in toners, preparations of magnetic pigments and the like.

Description of the Solubilization Method:

In the preparation of the solubilizates for cosmetic formulations, the copolymers according to the invention can be used as 100% strength substance or preferably as aqueous solution.

Usually, the solubilizer is dissolved in water and vigorously mixed with the sparingly soluble cosmetic active ingredient to be used in each case.

However, it is also possible to vigorously mix the solubilizer with the sparingly soluble cosmetic active ingredient to be used in each case and then to add demineralized water with continuous stirring.

Solubilizers for Pharmaceutical Applications:

The claimed copolymers are likewise suitable for use as solubilizer in pharmaceutical preparations of any type which are notable for the fact that they can comprise one or more medicaments which are insoluble or sparingly soluble in water, and also vitamins and/or carotenoids. In particular, these are solid solutions or solubilizates for oral application.

Thus, the claimed copolymers are suitable for use in oral administration forms such as tablets, capsules, powders, solutions. Here, they can make the sparingly soluble medicament available with increased bioavailability. Particular preference is given to using solid solutions of active ingredient and solubilizer.

In the case of parenteral application, it is also possible to use emulsions, for example fatty emulsions, besides solubilizates. The claimed copolymers are also suitable for this purpose, in order to process a sparingly soluble medicament.

Pharmaceutical formulations of the abovementioned kind can be obtained by processing the claimed copolymers with pharmaceutical active ingredients by conventional methods and with the use of known and novel active ingredients.

The use according to the invention can additionally comprise pharmaceutical auxiliaries and/or diluents. Cosolvents, stabilizers, preservatives are especially mentioned as auxiliaries.

The pharmaceutical active ingredients used are substances which are insoluble or slightly soluble in water. According to DAB 9 (German Pharmacopoeia), the solubility of pharmaceutical active ingredients is categorized as follows: slightly soluble (soluble in 30 to 100 parts of solvent); sparingly soluble (soluble in 100 to 1000 parts of solvent); virtually insoluble (soluble in more than 10 000 parts of solvent). The active ingredients can here come from any area of indication.

Examples which may be mentioned here are benzodiazepines, antihypertensives, vitamins, cytostatics, in particular taxol, anesthetics, neuroleptics, antidepressants, antibiotics, antimycotics, fungicides, chemotherapeutics, urologics, thrombocyte aggregation inhibitors, sulfonamides, spasmolytics, hormones, immunoglobulins, sera, thyroid therapeutic agents, psychopharmacological agents, antiParkinsonians and other antihyperkinetic agents, ophthalmics, neuropathy preparations, calcium metabolism regulators, muscle relaxants, narcotics, antilipemics, hepatic therapeutic agents, coronary agents, cardiacs, immunotherapeutics, regulatory peptides and their inhibitors, hypnotics, sedatives, gynecological agents, antigouts, fibrinolytic agents, enzyme preparations and transport proteins, enzyme inhibitors, emetics, circulation-promoting agents, diuretics, diagnostics, corticoids, cholinergics, bile duct therapeutics, antiasthmatics, broncholytics, beta-receptor blockers, calcium antagonists, ACE inhibitors, antiarterlosclerotics, anti-inflammatories, anticoagulants, antihypotensives, antihypoglycemics, antihypertonics, antifibrinolytics, antiepileptics, antiemetics, antidotes, antidiabetics, antiarrhythmics, antianemics, antiallergics, anthelmintics, analgesics, analeptics, aldosterone antagonists and slimming agents.

One possible preparation variant is to dissolve the solubilizer in the aqueous phase, if appropriate with gentle heating and then to dissolve the active ingredient in the aquaous solubilizer solution. The simultaneous dissolution of solubilizer and active ingredient in the aqueous phase is likewise possible.

The use of the copolymers according to the invention as solubilizer can, for example, also be carried out by dispersing the active ingredient in the solubilizer, if appropriate with heating, and mixing it with water with stirring.

In addition, the solubilizers can also be processed in the melt with the active ingredients. In particular, solid solutions can be obtained in this way. Of suitability for this is, inter alia, also the melt extrusion process. Another way of preparing solid solutions is also to prepare solutions of solubilizer and active ingredient in suitable organic solvents and then to remove the solvent by customary methods.

The invention thus also generally provides pharmaceutical preparations which comprise at least one of the copolymers according to the invention as solubilizer. Preference is given to those preparations which, besides the solubilizer, comprise a pharmaceutical active ingredient which is insoluble or sparingly soluble in water, for example from the abovementioned areas of indication.

Of the abovementioned pharmaceutical preparations, particular preference is given to those which are orally applicable formulations.

The content of solubilizer according to the invention in the pharmaceutical preparation is, depending on the active ingredient, in the range from 1 to 75% by weight, preferably 5 to 50% by weight, particularly preferably 10 to 30% by weight.

A further particularly preferred embodiment refers to pharmaceutical preparations in which the active ingredients and the solubilizer are present as solid solution. Here, the weight ratio of solubilizer to active ingredient is preferably from 1:1 to 4:1.

Solubilizers for Food Preparations:

Besides the use in cosmetics and pharmacy, the copolymers according to the invention are also suitable as solubilizers in the food sector for nutrients, auxiliaries or additives which are insoluble or sparingly soluble in water, such as, for example, fat-soluble vitamins or carotenoids. Examples which may be mentioned are clear drinks colored with carotenoids.

Solubilizers for Crop Protection preparations:

The use of the copolymers according to the invention as solubilizers in agrochemistry can comprise, inter alia, formulations which comprise pesticides, herbicides, fungicides or insecticides, especially also those preparations of crop protection compositions which are used as spray mixtures or pouring mixtures.

The copolymers according to the invention are notable for a particularly good solubilizing effect.

In the examples below, the preparation and use of the copolymers according to the invention are illustrated in more detail.

### EXAMPLES

To prepare the polymers, the following apparatus was used:

2 l apparatus with process-controlled waterbath, anchor stirrer and thermometer. The apparatus had connectors for 3 feeds, a reflux condenser and an inlet tube for introducing nitrogen or steam.

K values according to Fikentscher: 1% strength by weight solutions of the polymer in 0.1 mol of aqueous solution of NaCl.

Abbreviations used:

VCap: N-Vinylcaprolactam

VP: N-Vinypyrrolidone

LA: Lauryl acrylate

VL: Vinyl laurate

NaA: Sodium acrylate

### Example 1

Preparation of Copolymers of N-vinylcaprolactam/lauryl acrylate/sodium acrylate (Weight Ratio 85/5/10)

The initial charge was gassed with nitrogen and heated to a reactor internal temperature of 75° C. At a stirrer speed of 150 rpm, feed 1 and feed 2 were then introduced over the course of 4 hours, feed 3 was introduced over the course of 4.5 hours. The mixture was then after-polymerized for a further 2 hours at 75° C. 300 ml of ethanol were then distilled off and the reaction mixture was subjected to steam distillation. For this purpose, at an internal temperature of 102° C., 1 l of water was introduced as water vapor over a period of 1.5 hours. Following distillation, the polymer solution was diluted with 500 ml of water.

| | Amount g | | Substance |
|---|---|---|---|
| Initial charge | 350.0 | | Ethanol |
| | 10.0 | | VCap |
| Feed 1 | 400.0 | | Ethanol |
| | 330.0 | | VCap |
| | 20.0 | | LA |
| Feed 2 | 107.2 | g | 37.3% strength by weight |
| | | | solution of NaA in water |
| Feed 3 | 89.3 | | Ethanol |
| | 10.7 | | tertiary-Butyl perpivalate* |

This gave a clear, viscous solution. The K value was 17.1.

### Example 2

Preparation of Copolymers of N-vinylcaprolactam/lauryl acrylate/sodium acrylate (Weight Ratio 80/10/10)

The preparation was carried out analogously to Example 1.

| | Amount g | Substance |
|---|---|---|
| Initial charge | 350.0 | Ethanol |
| | 10.0 | VCap |
| Feed 1 | 400.0 | Ethanol |
| | 310.0 | VCap |
| | 40.0 | LA |
| Feed 2 | 107.2 | 37.3% strength by weight |
| | | solution of NaA in water |
| | 42.8 | Water |
| Feed 3 | 89.3 | Ethanol |
| | 10.7 | tertiary-Butyl perpivalate |

This gave a clear, viscous solution. The K value was 14.9.

### Example 3

Preparation of Copolymers of N-vinylcaprolactam/N-vinylpyrrolidone/vinyl laurate (Weight Ratio 60/30/10)

The initial charge of isopropanol and part of feed 1 was gassed with nitrogen and heated to a reactor internal temperature of 75° C. at a stirring speed of 75 rpm. Upon reaching an internal temperature of 73° C., part of feed 2 was added and the mixture was polymerized for 10 min. The remainder of feed 1 was then introduced over the course of 4 hours, and the remainder of feed 2 was introduced over the course of 5 hours. The mixture was then after-polymerized for a further 2 hours at 75° C. Then, isopropanol was distilled off and the reaction mixture was diluted with water and subjected to a steam distillation to give a solution with a solids content of 31.2% by weight. The K value was 13.5, measured at 1% strength by weight in water.

| | Amount g | Substance |
|---|---|---|
| Initial charge | 100.0 | Isopropanol |
| | 10.0 | VCap |
| | 75.0 | Feed 1 |
| | 5.33 | Feed 2 |
| Feed 1 | 250.0 | Isopropanol |
| | 300.0 | VCap |
| | 150.0 | VP |
| | 50.0 | VL |
| Feed 2 | 100.0 | Isopropanol |
| | 6.66 | tertiary-Butyl perpivalate |

This gave a clear, viscous solution. The K value was 14.9.
tertiary-Butyl perpivalate: 75% strength by weight active in aliphatics mixture, TBPPI-75-AL from Degussa, 82049 Puilach/Germany

For comparison, the following copolymers were prepared:

Comparative Example A

### Copolymer of N-vinylpyrrolidone/lauryl acrylate/sodium acrylate (Weight Ratio 80/10/10), K Value 13.5

### Comparative example B

### Copolymer of N-vinylpyrrolidone/lauryl acrylate/sodium acrylate (Weight Ratio 85/5/10), K Value 14.4

Preparation of Solid Solutions: General Procedure

To prepare the polymer/active ingredient mixture, the active ingredient and the polymer were weighed into a suitable glass vessel in the weight ratio 1:1 (in each case 2 g) and then 16 ml of dimethylformamide were added as solvent. The mixture was stirred at 20° C. for 24 hours on a magnetic stirrer. The solution was then drawn out using a 120 μm doctor knife on a glass plate. This was dried in the fume cupboard at RT for 0.5 hours and then dried in the drying cabinet at 50° C. and 10 mbar for a further 0.5 hours in order to remove all of the solvent. The samples were then assessed visually. If the films were clear and the active ingredient did not crystallize out after 7 days, the active ingredient was regarded as being stably dissolved within the polymer (data in Table 1: 50% dissolved). If no solid solution could be achieved using an active ingredient content of 50% by weight, the experiment was repeated using an active ingredient content of 33% by weight (data in Table 1: 33% dissolved). Overall, the copolymers according to the invention exhibited a higher capacity for forming a solid solution.

TABLE 1
Stability of a solid solution

| Copolymer | | | | |
|---|---|---|---|---|
| according to | Carbamazepine | Estradiol | Piroxicam | Clotrimazole |
| Ex. 1 | 50% dissolved | 50% dissolved | 33% dissolved | 50% dissolved |
| Ex. 2 | 33% dissolved | 50% dissolved | 50% dissolved | 50% dissolved |
| Ex. 3 | 50% dissolved | 50% dissolved | 33% dissolved | 50% dissolved |
| Comp. Ex. A | 33% dissolved | 33% dissolved | 33% dissolved | 33% dissolved |
| Comp. Ex. B | 33% dissolved | 33% dissolved | 33% dissolved | 33% dissolved |

Preparation of Solubilizates

2 g of the copolymer were weighed into a beaker. Then, one medicament in each case was weighed into the mixture as follows in order to obtain a supersaturated solution. (If the weighed-in mass dissolved in the medium, the initial weight was increased until a sediment formed).

Amount of active ingredient added: 17-R-estradiol 0.2 g; piroxicam 0.2 g; clotrimazole 0.2 g; carbamazepine 0.3 g

Phosphate buffer pH 7.0 was then added until solubilizer and phosphate buffer were present in the weight ratio 1:10. Using a magnetic stirrer, this mixture was stirred at 20° C. for 72 hours. There then followed a resting time of at least one hour. Following filtration of the mixture, it was measured photometrically and the content of active ingredient was determined.

The solubilizers according to the invention were significantly superior particularly in the case of the physiologically meaningful solubilization at 37° C.

TABLE 2
Solubilization at 20° C. in g/100 ml

| Copolymer | | | |
|---|---|---|---|
| according to | Carbamazepine | Estradiol | Piroxicam |
| Ex. 1 | 0.14 | 0.24 | 0.49 |
| Ex. 2 | 0.21 | 0.44 | 0.47 |
| Comp. ex. A | 0.11 | 0.12 | 0.28 |

TABLE 3
Solubilization at 37° C. in g/100 ml

| Copolymer | | | | |
|---|---|---|---|---|
| according to | Carbamazepine | Estradiol | Piroxicam | Clotrimazole |
| Ex. 1 | 0.26 | 0.22 | 0.75 | — |
| Ex. 2 | 0.37 | 0.36 | 0.59 | 0.12 |
| Comp. ex. A | 0.16 | 0.12 | 0.31 | — |

Determination of the Hygroscopicity

To determine the hygroscopicity, the weight increase of a sample of the copolymer was determined following storage for 24 hours at constant atmospheric humidity (76%) and checked again after 14 days. After 14 days, the values were unchanged. The copolymers according to the invention exhibited significantly lower hygroscopicity.

TABLE 4

| | Composition | | | | |
|---|---|---|---|---|---|
| | in [% by wt.] | | | | Weight increase |
| Copolymer according to | VP | VCap | LA | NaA | [%] |
| Ex. 1 | | 85 | 5 | 10 | 29 |
| Ex. 2 | | 80 | 10 | 10 | 27 |
| Comp. ex. A | 80 | | 10 | 10 | 47 |
| Comp. ex. B | 85 | | 5 | 10 | 52 |

1-27. (canceled)

28. A method comprising:
providing a substance selected from the group consisting of substances which are insoluble in water, substances which are only sparingly soluble in water, and combinations thereof; and
combining the substance and a solubilizing amount of a copolymer comprising:
(a) 60 to 99% by weight of N-vinylcaprolactam; and
(b) 1 to 40% by weight of at least one other monomer selected from the group consisting of: C₈-C₃₀-alkyl esters of monoethylenically unsaturated C₃-C₈-carboxylic acids; N—C₈₋₃₀ alkyl-substituted amides and. N,N—C₈.₃₀ dialkyl-substituted amides of acrylic acid, methacrylic acid, or combinations thereof; vinyl esters of aliphatic unbranched C₈-C₃₀-carboxylic acids; C₈-C₃₀-alkyl vinyl ethers; and mixtures thereof.

29. The method according to claim 28, wherein the copolymer further comprises up to 39% by weight of at least one further free-radically copolymerizable monomer selected from the group consisting of monoethylenically unsaturated carboxylic acids having 3 to 8 carbon atoms and salts thereof, esters of monoethylenically unsaturated C₃-C₈-carboxylic acids with C₁-C₄-mono- or dialcohols, nitriles of monoethylenically unsaturated C₃-C₈-carboxylic acids, N—C₁₋₄-alkyl- or N,N—C₁₋₄-dialkylamides of acrylic acid or methacrylic acid, N,N—C₁₋₄-dialkylamino-C₁₋₄-alkyl acrylates and salts thereof, acryl-amidoglycolic acid, monoethylenically unsaturated sulfonic acids, monoethylenically unsaturated monomers comprising phosphonic acid groups, vinyl acetate, N-vinylpyrrolidone, N-vinylimidazole and N-vinylformamide, wherein the weight percentages of N-vinylcaprolactam, the at least one other monomer and the at least one further free-radically copolymerizable monomer total 100%.

30. The method according to claim 29, wherein the N-vinylcaprolactam is present in the copolymer in an amount of 70 to 95% by weight, wherein the at least one other monomer is present in the copolymer in an amount of 5 to 30% by weight, and wherein the at least one further free-radically copolymerizable monomer is present in the copolymer in an amount of up to 25% by weight.

31. The method according to claim 29, wherein the at least one further free-radically copolymerizable monomer is present in the copolymer in an amount of 2 to 10% by weight.

32. The method according to claim 28, wherein the at least one other monomer comprises a C₈-C₃₀-alkyl ester of acrylic acid or methacrylic acid.

33. The method according to claim 28, wherein the at least one other monomer comprises lauryl acrylate.

34. The method according to claim 28, wherein the at least one other monomer comprises vinyl laurate.

35. The method according to claim 29, wherein the at least one further free-radically copolymerizable monomer comprises sodium acrylate.

36. The method according to claim 28, wherein the copolymer has a K value of 12 to 30.

37. The method according to claim 28, wherein the substance comprises at least one component selected from the group consisting of biologically active substances, pharmaceutical ingredients, cosmetic ingredients, agrochemical ingredients, food supplements, dietetic agents, foods, dyes, and combinations thereof.

38. A preparation comprising:
a substance selected from the group consisting of substances which are insoluble in water, substances which are only sparingly soluble in water, and combinations thereof; and
a solubilizing amount of a copolymer comprising:
(a) 60 to 99% by weight of N-vinylcaprolactam; and
(b) 1 to 40% by weight of at least one other monomer selected from the group consisting of: C₈-C₃₀-alkyl esters of monoethylenically unsaturated C₃-C₈-carboxylic acids; N—C₈₋₃₀ alkyl-substituted amides and N,N—C₈₋₃₀ dialkyl-substituted amides of acrylic acid, methacrylic acid, or combinations thereof; vinyl esters of aliphatic unbranched C₈-C₃₀-carboxylic acids; C₈-C₃₀-alkyl vinyl ethers; and mixtures thereof.

39. The preparation according to claim 38, wherein the copolymer further comprises up to 39% by weight of at least one further free-radically copolymerizable monomer, and wherein the weight percentages of N-vinylcaprolactam, the at least one other monomer and the at least one further free-radically copolymerizable monomer total 100%.

40. The preparation according to claim 39, wherein the at least one further free-radically copolymerizable monomer is selected from the group consisting of monoethylenically unsaturated carboxylic acids having 3 to 8 carbon atoms and salts thereof, esters of monoethylenically unsaturated C₃-C₈-carboxylic acids with C₁-C₄-mono- or dialcohols, nitriles of monoethylenically unsaturated C₃-C₈-carboxylic acids, N—C₁₋₄-alkyl- or N,N—C₁₋₄-dialkylamides of acrylic acid or methacrylic acid, N,N—C₁₋₄-dialkylamino-C₁₋₄-alkyl acrylates and salts thereof, acryl-amidoglycolic acid, monoethylenically unsaturated sulfonic acids, monoethylenically unsaturated monomers comprising phosphonic acid groups, vinyl acetate, N-vinylpyrrolidone, N-vinylimidazole and N-vinylformamide.

41. The preparation according to claim 38, wherein the substance and the copolymer are present in the form of a solid solution.

42. The preparation according to claim 38, wherein the substance comprises at least one component selected from the group consisting of biologically active substances, pharmaceutical ingredients, cosmetic ingredients, agrochemical ingredients, food supplements, dietetic agents, foods, dyes, and combinations thereof

43. The preparation according to claim 39, wherein the N-vinylcaprolactam is present in the copolymer in an amount of 70 to 95% by weight, wherein the at least one other monomer is present in the copolymer in an amount of 5 to 30% by weight, and wherein the at least one further free-radically copolymerizable monomer is present in the copolymer in an amount of up to 25% by weight.

44. The preparation according to claim 38, wherein the at least one other monomer comprises a C₈-C₃₀-alkyl ester of acrylic acid or methacrylic acid.

45. The preparation according to claim 38, wherein the at least one other monomer comprises lauryl acrylate.

46. The preparation according to claim 38, wherein the at least one other monomer comprises vinyl laurate.

47. The preparation according to claim 38, wherein the copolymer has a K value of 12 to 30.
